(19) United States
(12) Patent Application Publication
(10) Pub. No.: US 2026/0237063 A1
(43) Pub. Date: Aug. 13, 2026

(54) APPARATUS AND METHOD FOR REDUCTION OF MICRO-TISSUES FUNCTIONAL VARIABILITY, AND CONTROLLING THE EXPERIMENT FLOW BASED ON ML INFERENCE OF THE MICRO-TISSUES CONDITION

(71) Applicant: Quris Technologies Ltd., Tel Aviv (IL)

(72) Inventors: Isaac BENTWICH, Tel Aviv (IL); Yossi HARAN, Modi'in Makabim-Re'ut (IL); Shahar HAREL, Zikhron Ya'akov (IL)

(73) Assignee: Quris Technologies Ltd., Tel Aviv (IL)

(21) Appl. No.: 19/155,383

(22) PCT Filed: Feb. 13, 2024
(86) PCT No.: PCT/IL2024/050167

§ 371 Date: Aug. 11, 2025

Related U.S. Application Data
(60) Provisional application No. 63/445,334, filed on Feb. 14, 2023.

(51) Int. Cl. G06T 7/00; G01N 21/64; G01N 33/50

(52) U.S. Cl. CPC G06T 7/0012; G01N 21/6428; G01N 33/5005; G01N 2021/6439; G06T 2207/10056; G06T 2207/20081; G06T 2207/20084; G06T 2207/30024

(57) ABSTRACT

Systems and methods of non-invasive assessment of a three-dimensional cell culture, the method including receiving a training dataset comprising pairs of organoid images with a biochemical assay level, wherein the biochemical assay level is associated with a specific organoid and its corresponding image, training a deep learning regressor using the training dataset to determine a predicted biochemical assay level for a received image, receiving a new image of an organoid, applying the trained deep learning regressor to the received new image, and determining the predicted biochemical assay level for the received new image. The predicted biochemical assay can be used to compute non-invasive IC50 values and for variance reduction of organoid population.

### FIELD OF THE INVENTION

The present invention relates to non-invasive assessment of organoid viability. More particularly, the present invention relates to systems and methods for non-invasive, image analysis-based viability assessment of a three-dimensional cell culture, and its use for organoid variability reduction.

### BACKGROUND OF THE INVENTION

Adenosine triphosphate (ATP) is an organic compound that provides energy to drive many processes in living cells, and can be found in all forms of life. ATP is the energy source of all living cells and is involved in many vital biochemical reactions.

When cells die, they stop synthesizing ATP and the existing ATP pool is quickly degraded. Therefore, ATP is widely accepted as an indicator of cell viability. Higher ATP concentration indicates higher number of living cells.

To quantify ATP, cells are lysed to release the ATP for detection, and reagents containing firefly luciferase enzyme and substrate are added to catalyze a two-step reaction. Similar to ATP, other biochemical assays may be used, such as Lactate secretion. This endpoint assay comes with inherent limitation, since the lysis process breaks down the membrane of a cell, this marks the end of the organoid and thus require multiple biological repeats to track temporal phenomena and/or extract high frequency ATP levels.

It is known that 3-dimentional cell cultures (or organoids) exhibit functional variability. If not controlled, the variability can invalidate experiment results and/or analysis as the amount of variability obscures the signal at hand.

An organoid is a simplified version of an organ produced in vitro in three dimensions that shows realistic micro-anatomy. In order to measure ATP levels in an organoid, it is necessary to kill the cell to retrieve the required information (also known as an endpoint assay). For example, in a biological experiment on mice, it would be necessary to kill the mouse to get the results.

By measuring the ATP levels of a cell, researchers can gain insight into how the cell is functioning and whether or not it is healthy. This information can then be used to assess the safety of a medication, as well as the efficacy of different changes.

The half maximal inhibitory concentration (IC50) is a measure of the potency of a substance in inhibiting a specific biological or biochemical function. For example, it is conventionally used to determine drug potency with cell-based cytotoxicity tests (e.g., identifying the concentration resulting in 50% of the cell cytotoxic effect). IC50 curve is usually computed using multiple concentrations and their viability levels, where the curve is computed using logistic function fit (e.g., 3PL, 4PL).

It would therefore be advantageous to have a way to carry out long term testing on the organoids without killing them.

### SUMMARY OF THE INVENTION

A non-invasive assessment of organoid viability and the use of the viability assessment, among other organoid functional parameters, to reduce organoid population variability. More particularly, systems and methods are provided for non-invasive, image analysis-based viability assessment of a three-dimensional cell culture, and how it is used to reduce variance in organoid population, by using the viability assessment as a key functional parameter for clustering organoid into study groups.

There is thus provided, in accordance with some embodiments of the invention a method of non-invasive assessment of a three-dimensional cell culture, the method including: receiving, by a processor, a training dataset comprising pairs of an organoid image with a biochemical assay level, wherein the biochemical assay level is associated with a specific organoid and its corresponding image, training, by the processor, a deep learning regressor using the training dataset to determine a predicted biochemical assay level for a received image, receiving, by the processor, a new image of an organoid, applying, by the processor, the trained deep learning regressor to the received new image, and determining, by the processor, the predicted biochemical assay level for the received new image/organoid.

In some embodiments, the training dataset is acquired by imaging an organoid, and performing an assay on the organoid, to determine a biochemical assay level for that organoid. In some embodiments, performing the assay includes releasing the biochemical assay from the organoid, adding a reagent that glows in the presence of biochemical assay molecules, and measuring luminescent to determine a biochemical assay concentration.

In some embodiments, values of the received biochemical assay level are normalized for each image to a corresponding control group of organoids. In some embodiments, the deep learning regressor is trained on a resnet18 network with two additional fully connected layers, having an additional regression head, wherein the regressor outputs a predicted biochemical assay value.

In some embodiments, at least two organoids are grouped into a cluster based on common functional parameters, comprising the biochemical assay, and the method further comprising using the clustering to re-group organoids. In some embodiments, each cluster is associated with different organoids having similar functional properties.

In some embodiments, the biochemical assay is an Adenosine triphosphate. In some embodiments, the biochemical assay is a Lactate. In some embodiments, the determined biochemical assay is a viability assessment assay.

There is thus provided, in accordance with some embodiments of the invention a system for non-invasive assessment of a three-dimensional cell culture, the system including: a database, comprising training dataset comprising pairs of an organoid image with a biochemical assay level, wherein the biochemical assay level is associated with a specific organoid and its corresponding image, and a processor, coupled to the database, wherein the processor is configured to: train a deep learning regressor using the training dataset to determine a predicted biochemical assay level for a received image, receive a new image of an organoid, apply the trained deep learning regressor to the received new image, and determine the predicted biochemical assay level for the received new image.

In some embodiments, the system includes an imager (e.g., a microscope), coupled to the processor, and configured to image an organoid, wherein the training dataset is acquired by the processor performing an assay on the organoid, to determine a biochemical assay level for that organoid. In some embodiments, performing the assay includes: releasing the biochemical assay from the organoid, adding a reagent that glows in the presence of biochemical assay molecules, and measuring luminescent to determine a biochemical assay concentration.

In some embodiments, the processor is configured to normalize values of the received biochemical assay level for each image to a corresponding control group of organoids. In some embodiments, the deep learning regressor is trained on a resnet18 network with two additional fully connected layers, having an additional regression head, wherein the regressor outputs a predicted biochemical assay value.

In some embodiments, at least two organoids are grouped into a cluster based on common functional parameters, comprising the biochemical assay, and the processor is configured to use the clustering to re-group the organoids. In some embodiments, each cluster is associated with a different organoid having similar functional properties. In some embodiments, the biochemical assay is an Adenosine triphosphate. In some embodiments, the biochemical assay is a Lactate. In some embodiments, the determined biochemical assay level is a viability assessment assay.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a block diagram of a computing device, according to some embodiments of the invention;

FIGS. 2A-2B show a block diagram of a system for non-invasive assessment of a three-dimensional cell culture, according to some embodiments of the invention;

FIG. 3 shows some experimental results versus prediction of ATP level of the system for non-invasive assessment of a three-dimensional cell culture, according to some embodiments of the invention;

FIGS. 4A-4B show a flowchart for a method of non-invasive assessment of a three-dimensional cell culture, according to some embodiments of the invention; and

FIG. 5 shows a flowchart for a method of variability reduction using functional-clustering using the system for non-invasive assessment, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details.

In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items.

Unless explicitly stated, the method embodiments described herein are not constrained to a particular order or sequence. Additionally, some of the described method embodiments or elements thereof may occur or be performed simultaneously, at the same point in time, or concurrently.

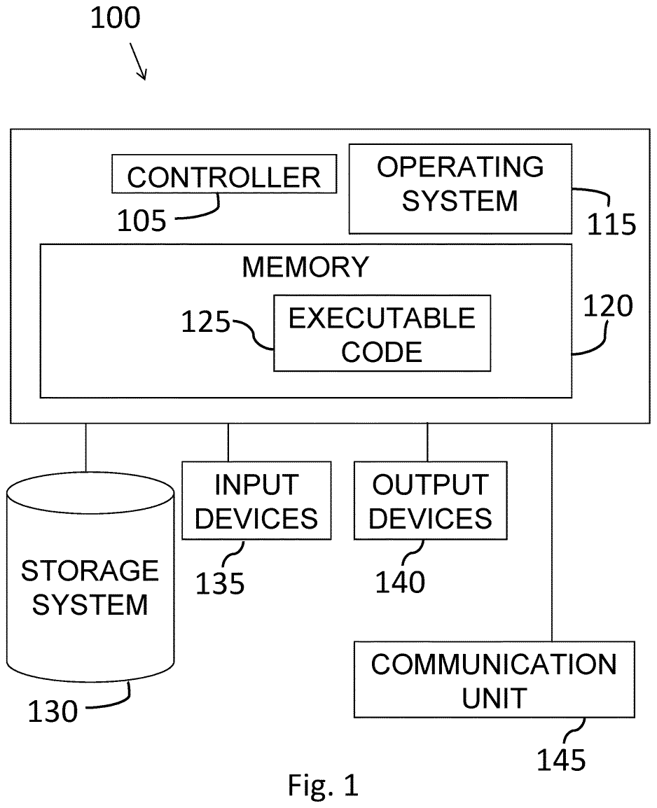

Reference is made to FIG. 1, which is a block diagram of an example computing device, according to some embodiments of the invention. Computing device 100 may include a controller or processor 105 (e.g., a central processing unit processor (CPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet.

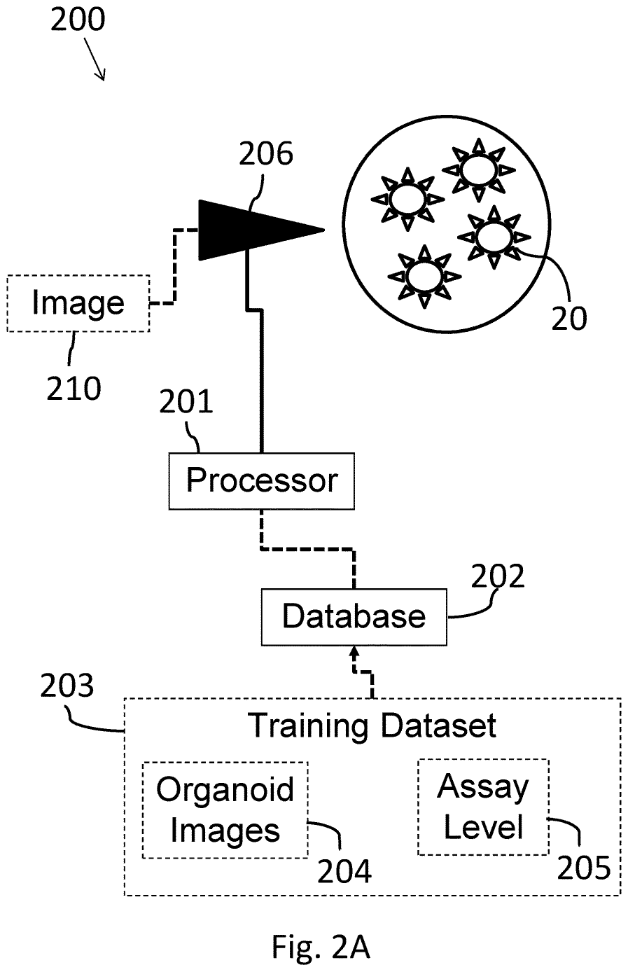

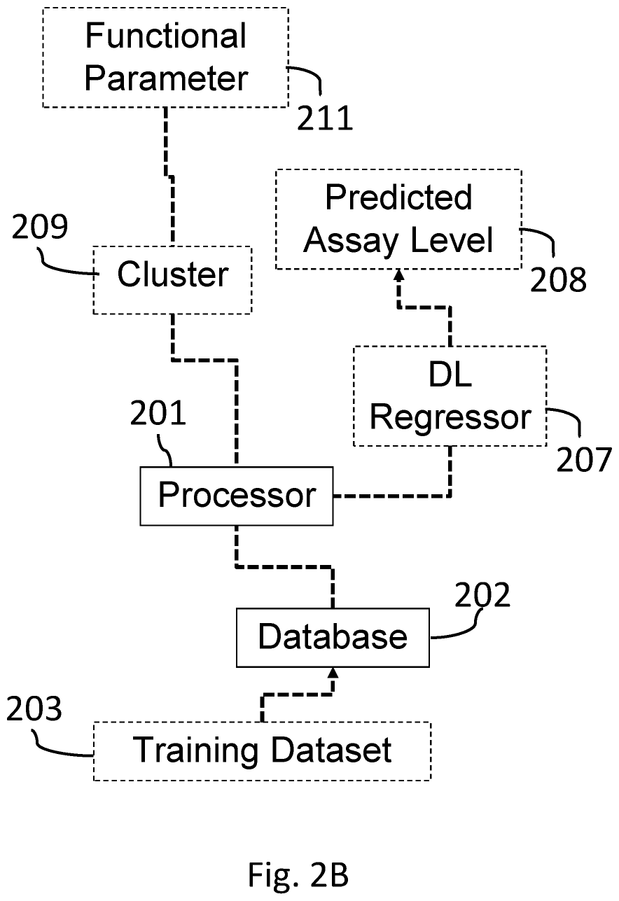

Controller 105 may be configured to execute program code to perform operations described herein. The system described herein may include one or more computing device(s) 100, for example, to act as the various devices or the components shown in FIG. 2A. For example, communication system 200 may be, or may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of similar and/or different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein.

Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 are omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100.

Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105.

Such a non-transitory computer readable medium may be for example a memory, a disk drive, or a USB flash memory, encoding, including or storing instructions, e.g., computer-executable instructions, which when executed by a processor or controller, carry out methods disclosed herein.

The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPUs), a plurality of graphics processing units (GPUs), or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components.

In some embodiments, a system may include or may be, for example, a personal computer, a desktop computer, a laptop computer, a workstation, a server computer, a network device, or any other suitable computing device.

For example, a system as described herein may include one or more facility computing device 100 and one or more remote server computers in active communication with one or more facility computing device 100 such as computing device 100, and in active communication with one or more portable or mobile devices such as smartphones, tablets and the like.

According to some embodiments, systems and methods are provided for non-invasive viability assessment for organoids. The organoid may be imaged to retrieve the required information, without the need to kill the organoid.

The retrieved information may accordingly be used to group and/or reduce the organoid population variability.

For example, in an experiment performed on organoids to test the response to a particular medication, the long terms affects of the medications may only be analyzed by not killing the organoids, that is in contrast to commercially available methods.

Accordingly, viability-based clustering into study groups (e.g., compared to a reference group of organoids that do not receive medication treatment) may only be achieved by not killing the organoids, thereby providing a new way to study these organoids.

Reference is now made to FIGS. 2A-2B, which shows a block diagram of a system 200 for non-invasive assessment of a three-dimensional cell culture, according to some embodiments of the invention. In FIGS. 2A-2B, hardware elements are indicated with a solid line and the direction of arrows indicate a direction of information flow between the hardware elements.

The system 200 may include a processor 201 (e.g., such as the controller 105 shown in FIG. 1) that is coupled to a database 202 (e.g., such as the storage system 130 shown in FIG. 1).

The database 202 may include a training dataset 203 including pairs of organoid images 204 with a biochemical assay level 205. The biochemical assay level 205 may be associated with a specific organoid 20 and its corresponding image 204.

The training may include splitting the data into train/validation/test sets of sizes 60/20/20 percent, respectively. The train set may be accordingly used for optimizing the model parameters, with the validation to early stop the training and for hyper parameter optimization, and the test set for the final evaluation only.

For example, a plurality of organoids 20 may be positioned on dedicated plate and continuously imaged by a dedicated imager 206.

The imager 206 (e.g., a high-resolution microscope) may be coupled to the processor 201, such that the processor 201 may receive the organoid images from the imager 206 (e.g., a brightfield image).

For example, brightfield images may be acquired on an ECHO Rebel microscope using these settings: 22% light intensity, 72% brightness, 100% contrast, 33% white balance and ×10 objective.

In some embodiments, the biochemical assay level may be an ATP level. In an initial extraction stage, the ATP assay level may be determined by releasing the biochemical assay from the organoid, adding a reagent that glows in the presence of biochemical assay molecules, and measuring luminescent to determine a biochemical assay concentration.

The processor 201 may for instance automatically collect and estimate multiple indications of organoid healthiness and/or functionality (e.g., for the first 24 hours of the experiment).

The functionality properties may include organ functional biological and/or chemical markers (e.g., for a liver, using image-derived viability assessment, levels of Albumin, UREA, Glucose, and morphology parameters such as organ size and sphereness), as well as the estimated viability.

In some embodiments, the processor 201 is configured to train a deep learning (DL) regressor 207 using the training dataset 203 to determine a predicted biochemical assay level 208 for a received image 210.

For example, the processor 201 may generate thousands of pairs. A pair per organoid 20, may include the image 210 and the corresponding biochemical assay level 208.

The processor 201 may receive a new image 210 of the organoid 20, for instance received from the imager 206. Thus, the DL regressor 207 may predict the biochemical assay level 208 for a new image 210 of an organoid 20, so that there is no longer a need to kill that organoid 20. For example, the result of such training may generate R-squared being larger than 0.85.

For example, the DL regressor may be trained on a resnet18 network with two additional fully connected layers, and an additional regression head, in order to output a single number for the predicted biochemical assay value 208.

After training, the organoids 20 may be grouped or clustered according to their functional properties. For example, clustering into equal size clusters depending on the number of organoids.

The clustering is carried out using k-means algorithm in a first phase, and equalizing the cluster size in a second phase, by defining cluster uniqueness score per organoid as its distance to cluster centroid divided by its distance to 2nd closest cluster centroid (or divide by the average distance to all cluster centers), and sort clusters from the largest to smallest, then iterate and replace organoid assignment from the large clusters to smaller cluster according to the uniqueness scores (move organoid with high score to the next closest available center) until the cluster reach the desired size.

In some embodiments, study groups (or clusters) are generated to maximize the similarity between study groups, by selecting each study group to include an organoid from each functional cluster so that each study group accordingly includes a representative from each organoid functionality.

At least two organoids 20 may be grouped into a cluster 209 based on a common functional parameter 211.

In some embodiments, a dedicated device may move organoids in accordance with the results of a grouping algorithm, so that the organoids are dispersed on a biochip based on their assigned cluster.

For example, each biochip may also include organoids serving as control group, the control group not being treated with any medication and serve as normalizer for the organoid in the same biochip.

In some embodiments, values of the received biochemical assay level 208 may be normalized for each image 210 to a corresponding control group of organoids 20.

For example, after the grouping phase, the functional variability between the groups may be observed as 17 times lower. In another example, random grouping may have substantially larger variability than the functional grouping so that clustering based on functional properties may generate system that has optimized signal to noise ratio, for instance compared to commercially available solutions and therefore provides accurate results.

According to some embodiments, the processor 201 applies the trained deep learning regressor 207 to the received new image 210 in order to predict and determine the biochemical assay level 208 for the received new image 210.

Using the predicted biochemical assay level 208 it may therefore be possible to get high frequency assay level data for each organoid 20 during future experiments. Accordingly, the system 200 provides a way for long term experiments on the organoids, as there is no longer a need to kill the organoids.

In some embodiments, similar processes may be carried out with other biological indications (such as the ATP level), and may accordingly prevent invasive tests on organoids/animals/humans.

For example, the IC50 computation is carried out using the invasive ATP assay, so that multiple organoids must be killed per concentration to estimate the cytotoxic effect. Using the ATP estimation method as provided by system 200, it is possible to determine IC50 using organoid images only.

In some embodiments, population of organoids is treated with multiple dosages of a medication and the organoids are imaged on a daily basis, where for each image the ATP level is predicted. Using the predicted ATP level, killing the organoid is no longer required, since the dose-response graph may be generated from the predicted (image based) values, for instance using logistic curve fit (e.g., 3PL, 4PL). Thus, it is possible to identify the IC50 in a non-invasive manner. In some embodiments, the IC50 may be determined on a daily basis, which better describes the cytotoxic effect over time, and allows better decision making and better feature generation (e.g., for ML models) For example, having estimated non-invasive viability assessment allows to dynamically evaluate and change the experiment in real-time. For example, the frequency of sampling biomarkers is adjusted according to estimated viability levels (e.g., if predicted viability present significant changes in recent time, we will sample biomarkers with higher frequency and vice versa), to provide better resolution in times of significant changes.

Generating an image for explaining the viability prediction by highlighting the areas (pixels) in the image that contribute mostly to the level of viability. This may be accomplished by attributing the gradients of the deep machine learning algorithm to the input image. Scores are assigned to pixels according to the gradient magnitude versus a reference image. The explanation image is a type of “AI staining”, used to better diagnose the organoid and better characterize the organoid condition.

AI staining may be used both for human operator visibility to better understand the reasoning behind the deep learning algorithm predictions (e.g., as a decision assistance tool for the operator) as well as by further algorithms, using the generated AI staining as features and/or as input to a downstream task (e.g., for toxicity prediction).

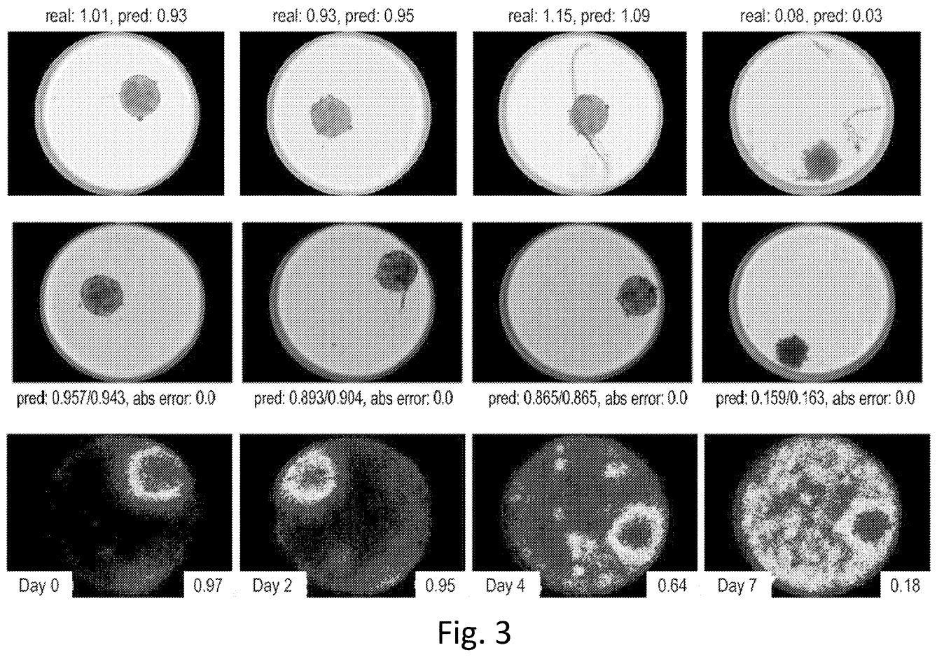

Reference is now made to FIG. 3, which shows some experimental results versus prediction of ATP level of the system for non-invasive assessment of a three-dimensional cell culture, according to some embodiments of the invention.

In this experiment, that is carried out after the training stage, the organoid was treated with a toxic drug along 7 days of experiment. The images in FIG. 3 show time series of the organoid images, as well as time series of explanations, with spheroid biomarker (e.g., viability) prediction based on a brightfield image, and AI staining where staining of the morphological areas corresponds to the biomarker (e.g., indicating degradation in viability).

It may be observed that real versus predicted ATP levels show high correlation, and predicted ATP is correctly detecting degradation in the cell viability, and being correctly estimated by the vision-based regressor.

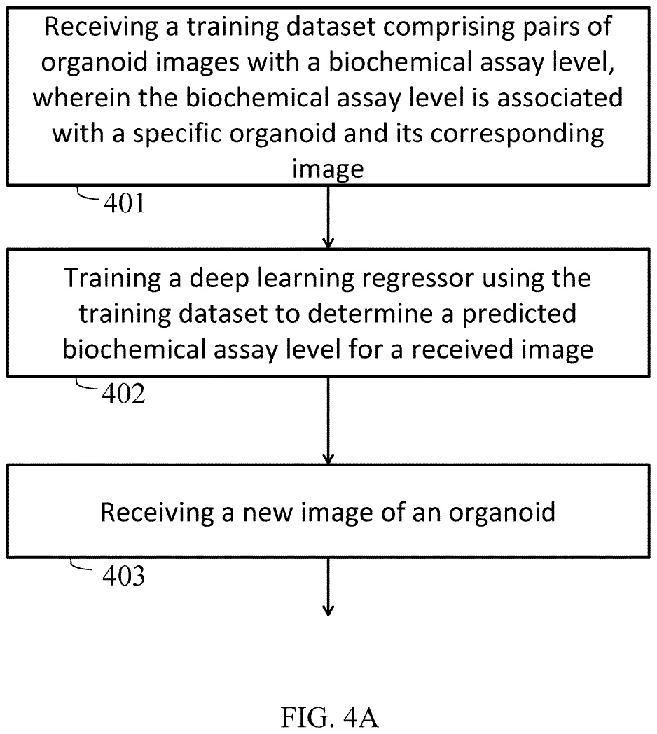

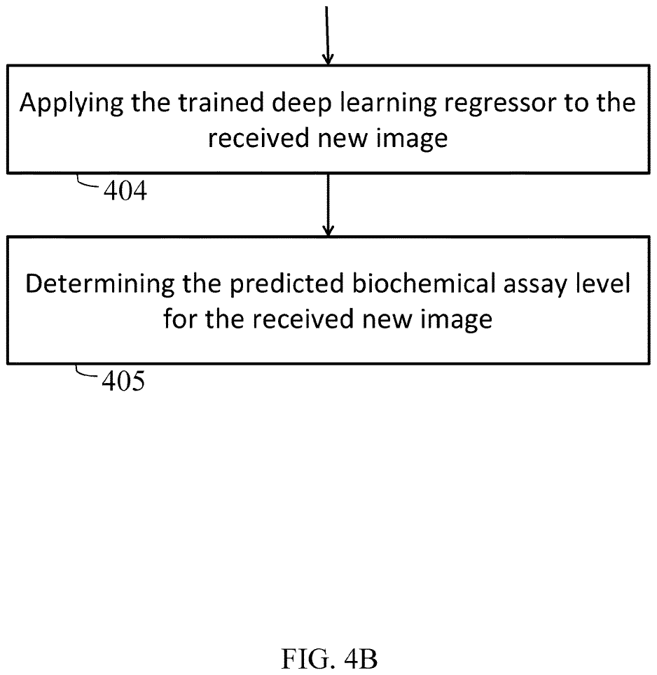

Reference is now made to FIGS. 4A-4B, which show a flowchart for a method of non-invasive assessment of a three-dimensional cell culture, according to some embodiments of the invention.

The processor receives 401 a training dataset comprising pairs of organoid images with a biochemical assay level, wherein the biochemical assay level is associated with a specific organoid and its corresponding image.

The processor trains 402 a deep learning regressor using the training dataset to determine a predicted biochemical assay level for a received image.

The processor receives 403 a new image of an organoid.

The processor applies 404 the trained deep learning regressor to the received new image.

The processor determines 405 the predicted biochemical assay level for the received new image.

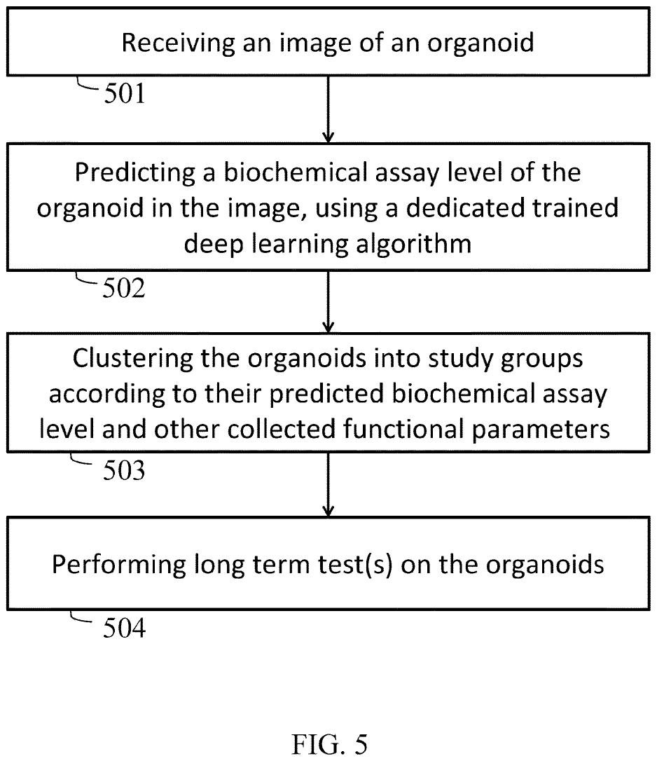

Reference is now made to FIG. 5, which shows a flowchart for a method of variability reduction using functional-clustering using the system for non-invasive assessment, according to some embodiments of the invention, as well as other functional parameters.

According to some embodiments, with the system for non-invasive assessment (e.g., as described above), long term test may be viable since there is no longer a need to kill the organoids.

An image of organoid(s) may be received 501, and based on the dedicated trained deep learning algorithm, the biochemical assay level of the organoid(s) may be predicted 502. The organoid(s) may be clustered 503, into study groups according to their predicted biochemical assay level and other collected functional parameters.

Accordingly, long-term test may be performed 504 on these organoid(s), since there is no longer a need to kill them, and s comparison to a reference group of organoids may be achieved.

While certain features of the invention have been illustrated and described herein, many modifications, substitutions, changes, and equivalents may occur to those skilled in the art. It is, therefore, to be understood that the appended claims are intended to cover all such modifications and changes.

Various embodiments have been presented. Each of these embodiments may of course include features from other embodiments presented, and embodiments not specifically described may include various features described herein.

1. A method of non-invasive assessment of a three-dimensional cell culture, the method comprising:
receiving, by a processor, a training dataset comprising pairs of an organoid image with a biochemical assay level, wherein the biochemical assay level is associated with a specific organoid and its corresponding image;
training, by the processor, a deep learning regressor using the training dataset to determine a predicted biochemical assay level for a received image;
receiving, by the processor, a new image of an organoid;
applying, by the processor, the trained deep learning regressor to the received new image; and
determining, by the processor, the predicted biochemical assay level for the received new image.

2. The method of claim 1, wherein the training dataset is acquired by:
imaging an organoid; and
performing an assay on the organoid, to determine a biochemical assay level for that organoid.

3. The method of claim 2, wherein performing the assay comprises:
releasing the biochemical assay from the organoid;
adding a reagent that glows in the presence of biochemical assay molecules; and
measuring luminescent to determine a biochemical assay concentration.

4. The method of claim 1, further comprising normalizing values of the received biochemical assay level for each image to a corresponding control group of organoids.

5. The method of claim 1, wherein the deep learning regressor is trained on a resnet18 network with two additional fully connected layers, having an additional regression head, wherein the regressor outputs a predicted biochemical assay value.

6. The method of claim 1, wherein at least two organoids are grouped into a cluster based on common functional parameters, comprising the biochemical assay, and the method further comprising using the clustering to re-group organoids.

7. The method of claim 6, wherein each cluster is associated with different organoids having similar functional properties.

8. The method of claim 1, wherein the biochemical assay is an Adenosine triphosphate.

9. The method of claim 1, wherein the biochemical assay is a Lactate.

10. The method of claim 1, wherein the determined biochemical assay is a viability assessment assay.

11. A system for non-invasive assessment of a three-dimensional cell culture, the system comprising:
a database, comprising training dataset comprising pairs of an organoid image with a biochemical assay level, wherein the biochemical assay level is associated with a specific organoid and its corresponding image; and
a processor, coupled to the database, wherein the processor is configured to:
train a deep learning regressor using the training dataset to determine a predicted biochemical assay level for a received image;
receive a new image of an organoid;
apply the trained deep learning regressor to the received new image; and
determine the predicted biochemical assay level for the received new image.

12. The system of claim 11, further comprising an imager, coupled to the processor, and configured to image an organoid, wherein the training dataset is acquired by the processor performing an assay on the organoid, to determine a biochemical assay level for that organoid.

13. The system of claim 12, wherein performing the assay comprises:
releasing the biochemical assay from the organoid;
adding a reagent that glows in the presence of biochemical assay molecules; and
measuring luminescent to determine a biochemical assay concentration.

14. The system of claim 11, wherein the processor is configured to normalize values of the received biochemical assay level for each image to a corresponding control group of organoids.

15. The system of claim 11, wherein the deep learning regressor is trained on a resnet18 network with two additional fully connected layers, having an additional regression head, wherein the regressor outputs a predicted biochemical assay value.

16. The system of claim 11, wherein at least two organoids are grouped into a cluster based on common functional parameters, comprising the biochemical assay, and the processor is configured to use the clustering to re-group the organoids.

17. The system of claim 16, wherein each cluster is associated with a different organoid having similar functional properties.

18. The system of claim 11, wherein the biochemical assay is an Adenosine triphosphate.

19. The system of claim 11, wherein the biochemical assay is a Lactate.

20. The system of claim 11, wherein the determined biochemical assay level is a viability assessment assay.
